(19) United States
(12) Patent Application Publication
(10) Pub. No.: US 2007/0191730 A1
(43) Pub. Date: Aug. 16, 2007

(54) VIBROTACTILE PERCEPTION METER

(75) Inventor: Antonio Speidel, S-230 42 Tygelsjo (SE)

Correspondence Address: LARSON AND LARSON, 11199 69TH STREET NORTH, LARGO, FL, 33773, US

(73) Assignee: VIBROSENSE DYNAMICS AB, Malmo (SE)

(21) Appl. No.: 11/737,909

(22) Filed: Apr. 20, 2007

Related U.S. Application Data
(63) Continuation-in-part of application No. PCT/SE05/01450, filed on Oct. 3, 2005.

(30) Foreign Application Priority Data
Oct. 25, 2004 (SE) 0402569-8

(51) Int. Cl. A61B 5/00

(52) U.S. Cl. 600/552

(57) ABSTRACT

Identifying a vibrotactile perception threshold of different mechanoreceptors at a skin site of a subject by detecting sensory deficits in peripheral nerves and, in particular, those associated with neuropathy in the fingers or other parts of the body. Controlling and monitoring of the static dynamic contact force between the human skin site and a vibrating probe which transmits a frequency signal to a receiver for measuring the spatial position of the transmitted signal, whereby a human feedback device records the signal and adjusts for continuous contact force between the skin site and vibrating probe.

### PRIOR APPLICATIONS

This application is a continuation-in-part of International Application No. PCT/SE2005/001450, filed Oct. 3, 2005, which in turn bases priority on Swedish Application No. 0402569-8, filed Oct. 25, 2004.

### BACKGROUND OF THE INVENTION

1. Field of Invention

The invention relates, generally, to an apparatus for detecting sensory deficits in peripheral nerves and, in particular, to an apparatus for detecting sensory deficits associated with neuropathy in the fingers or other parts of the body.

More particularly, this invention relates to an apparatus for identifying vibrotactile perception thresholds of different mechanoreceptors at a skin site of a subject to assess sensory change in tactile sensory nerve function, and wherein the resultant threshold signals obtained by the method are substantially void of errors or inconsistencies.

2. Description of the Prior Art

Many provocative tests have long been used to detect the very early sensory impairment which usually occurs in diabetes neuropathy and neuropathy caused by vibration exposure. These tests include the “two-point discrimination” test for tactile gnosis, and the monofilament test for touch/pressure. However, for various reasons, these tests have been found to be ineffective in early stages of neuropathy. On the contrary, the vibration sense of the hand is very early impaired in metabolic or vibration-induced neuropathy, and various tests on vibration perception of the hand have, therefore, been developed. However, the effectiveness of the vibratory tests is dependent on many test parameters, such as frequency, dimensions of the vibrating rod or probe area and, very important, the “probe contact force.”

The measurement technique involves supporting the body part containing the skin site to be studied, and stimulating the skin surface with vibration under controlled contact conditions in such a way that a single mechanoreceptor population mediates the threshold at each frequency. Accordingly, it is a principal objective of the present invention to provide a screening or diagnostic system to measure the extent of sensory disturbances in neuropathies or any response to treatment of any such sensory disturbance. These objectives are attained, generally, in a system to sense a body pressure sensitivity phenomenon of a patient that includes a vibratory stimulator to apply controlled and compensated vibratory force to a finger or other body part of the patient, a drive mechanism connected to effect vibration of the vibratory stimulator.

In U.S. Pat. No. 5,002,065 to LaCourse, et al, a method is described on how to achieve a well-controlled amplitude and acceleration on the probe by utilizing a closed loop control system. However, this means that the amplitude and acceleration will be independent of the applied contact force on the vibrating probe. The used backpressure monitor measures the acceleration on the vibrating probe without any consideration to the applied contact force which considerably degrades testing reliability.

In U.S. Pat. No. 5,433,211 to Brammer, et al, the applied contact force on the vibrating probe is controlled by an external complex mechanical counter weight mechanism without actually measuring the applied contact force. This complexity increases the possibility of erroneous test set up conditions without any control of the test environment, i.e. the measured vibrotactile perception thresholds (VPT) may be recorded with incorrect applied contact force without any notice which degrades testing reliability.

U.S. Pat. No. 5,673,703, to Fisher, et al, describes an apparatus for automatic testing of vibrotactile responses of a patient. In the preferred embodiment of the invention, a general purpose computer functions to control the operation of the system, and to record and store the patient's responses. Indentations and vibrations are produced by off-axis rotation of a stimulation probe. A frequency-modulated signal generated by the computer is used to control a motor, which drives the stimulation probe. This apparatus falls short since changes in the contact force will affect both the motor speed (frequency) and the amplitude for the stimulus probe. In fact, the described principle for generating the probe movement will measure vibrotactile perception thresholds (VPT) with a very low precision and accuracy due to the inferior control mechanism and test set up. Thus, the detected VPT will strongly vary depending on the applied contact force that is not controlled in any way and, accordingly, degrades testing reliability.

In WO 0059377 A1 to LaCourse, et al, the applied contact force is measured indirectly by measuring the applied force on a surround at which the finger rests during the test. This method requires more complex test equipment and the required applied contact force is much larger compared to when measuring without any surround. A higher contact force will also require a stronger (larger) vibrator that consumes more power which will further increase both the physical weight and the manufacturing cost for the device (instrument).

### SUMMARY OF THE INVENTION

The object of the invention is to provide a means to control and monitor the static and dynamic contact force between human skin and a vibrating probe. This is very important when measuring the Vibrotactile Perception Thresholds (VPT) in order to get accurate test conditions set up to achieve required measurement precision.

The invention is a screening or diagnostic testing apparatus, namely, a system and a method of said screening for peripheral neuropathies. In its most basic form, the apparatus includes a surface having an opening, a surround disposed around the opening for a vibrating rod disposed within said opening for contact with the pulp of a finger or other body part. The preferred apparatus includes a pressure sensor for sensing a pressure exerted by the body part upon the probe to ensure that pressure applied to the body part is within a specified range, and means for ensuring said continuous contact with the body part.

In the context of the present application and invention, the following definitions apply:

VPT=Vibrotactile Perception Threshold

RFD=Requested Force Displacement

RF=Requested Force

CF=Contact Force

SI=Sensibility Index

rms=Root Mean Square

### BRIEF DESCRIPTION OF THE DRAWINGS

Further features and advantages of the present invention can be gathered from the following descriptions of the preferred embodiment with reference to the attached drawings, wherein:

FIG. 1 illustrates the force control system;

FIG. 2 illustrates force and spatial positions of the force control system;

FIG. 3 illustrates the required detector signal in an unbalanced system;

FIG. 4 illustrates the required detector signal in a balanced system.

### DETAILED DESCRIPTION OF THE PREFERRED EMBODIMENT

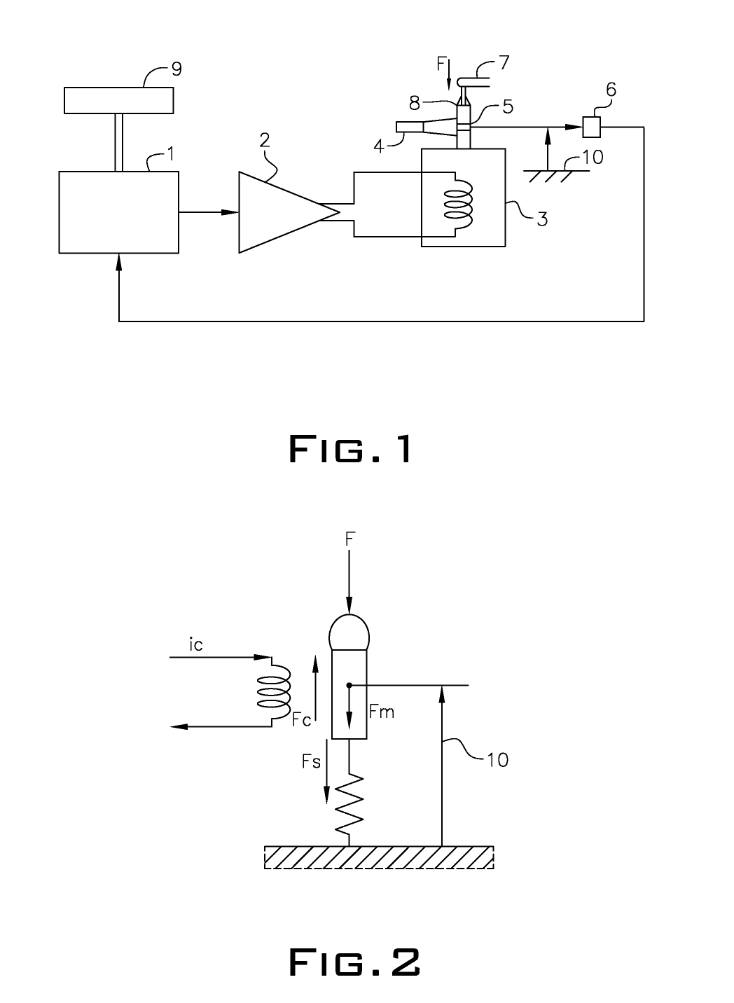

Referring to FIG. 1, a force control system discloses a micro computer system 1 comprising a microprocessor and interface electronics with AD and DA-converters. Further disclosed is an amplifier 2 which amplifies an analog signal from the micro computer system 1, the amplified signal drives the electro dynamic vibrator 3. Said vibrator is an electro dynamic device with an attached probe 8 which is moving when a current or a voltage is applied to said device. Transmitter 4 is a transmitting device which sends out some kind of signal, i.e. an optical beam (light) or an electrical or magnetic field. Aperture 5 is a device, i.e. a hole or a lens, which limits or focuses the transmitted signal in space. The aperture 5 is optional and is not required if the transmitted signal is narrow enough. Detector 6 is a device that detects static or a dynamic spatial position 10 of the vibrating probe 8 by measuring the transmitted signal from the transmitter 4 in an appropriate manner. A human body part 7, i.e. a finger or a toe, is pressed with the force F against the vibrating probe 8. A Vibrating probe 8 comprises a probe that is fixedly attached to the moving part, i.e. a membrane in the electro dynamic vibrator 3. A human feedback device 9 is used by the micro computer system 1 to report the displacement in the position caused by the force F. The spatial position 10 is relative to a fixed reference point of origin.

FIG. 2 shows forces F and spatial positions 10 where i_{c} is the current through the electro dynamic vibrator 3, F_{c} is the probe 8 force created by the current i_{c} supplied to the electro dynamic vibrator 3, Fₛ shows the spring force created by the probe 8 offset inside the electro dynamic vibrator 3, m is the moving mass (probe+membrane) in the electro dynamic vibrator 3, Fₘ shows the gravitational force on the moving mass m, F is the external force cause by a calibration force (weight) or by a pressure from a body part 7, and X is the spatial position 10 relative to a fixed reference point of origin.

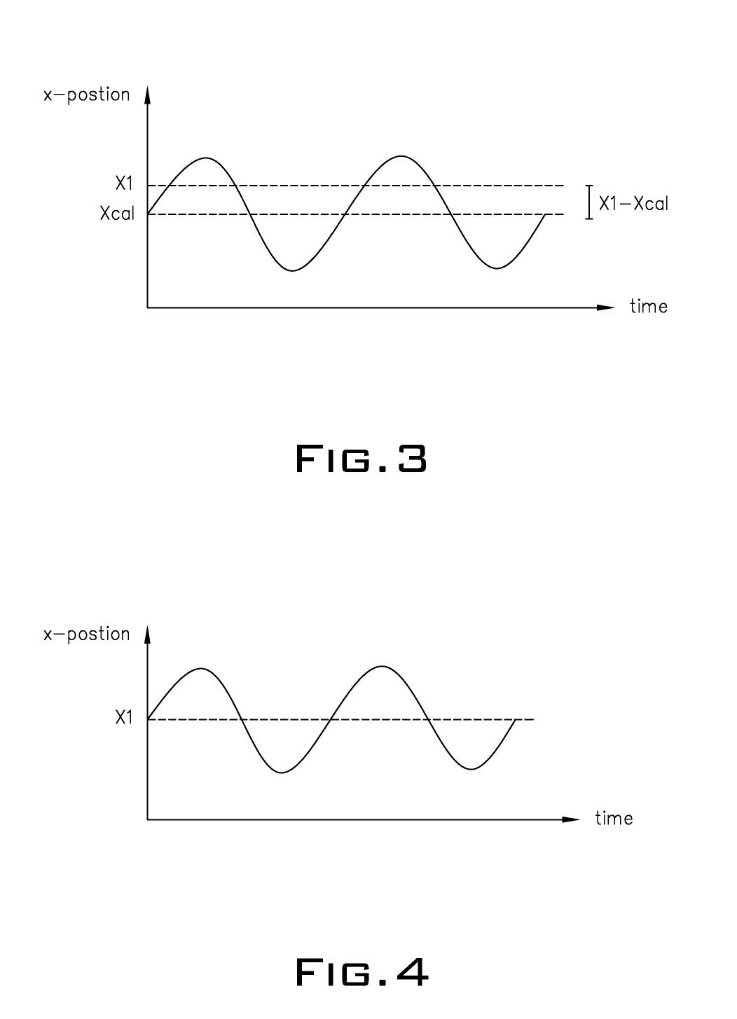

FIG. 3 shows the required detector signal in an unbalanced system where X₁ is the spatial position 10 for an unloaded system, and X_{cal} is the spatial position 10 when the calibration weight is mounted on the vibrating probe 8 without added DC-current i_{c}, i.e. when i_{c}=0.

The detection principle is shown in FIG. 1, whereby the contact force F is created by the patient by pressing the body part 7 to be examined against the vibrating probe 8. The patient controls the applied force F by adjusting the force in accordance with the reading on the output presented by the human feedback device 9. The correct force is then applied when the human feedback device 9 presents a predetermined condition, i.e. correct color, sound, or numerical value.

The applied force F is measured indirectly by measuring the change of the spatial position 10 on the vibrating probe 8 as a static displacement caused by the force F. Since the vibrating probe 8 is mounted in a spring supported mechanical construction, any displacement corresponds to a specific force in a linear fashion. Therefore, the displacement will be an indirect measure of the applied force F, i.e. the force may be calculated by measuring the occurred static displacement of the spatial position 10.

The unloaded electro dynamic vibrator 3, without an applied external force F, may be calibrated by adding a well-known force, the requested force RF, i.e. a calibration weight. The occurred displacement for the unloaded electro dynamic vibrator 3, the difference in the spatial position 10 of the probe 8 with and without the calibration weight, will then correspond to a specific force.

The displacement caused by the calibration weight is denoted as the Requested Force Displacement RFD. During normal operation, the RFD may be used as a requested absolute static offset which should be maintained during the complete test cycle.

A contact force F below or above the RF will be presented on the human feedback device 9 as a “too low value” or a “too high value”, respectively. On the human feedback device 9, the RFD may be visualized on a bar graph array as the center value.

The displacement is measured by the detector 6 which detects the signal emanating from the transmitter 4 passing the optional aperture 5. One or two of these items, the transmitter 4, aperture 5 or the detector 6, may be located directly on the vibrating probe 8, whereas at least one item should be spatially fixed.

The output signal from the detector 6 corresponds to a spatial position 10 of the probe 8. This signal may be processed, filtered and then converted to a digital signal (DA-conversion) within the micro computer system 1. The digital signal is read by a microprocessor, which is part of the micro computer system 1. The microprocessor compares the read digital signal caused by the contact force F with a previously stored value for the signal caused by the calibrating force RF and outputs the difference on the human feedback device 9.

Instead of maintaining a calibrated static offset for the spatial position 10, the offset may be outbalanced by applying an overlaid calibrated DC-current i_{c} in the electrical current signal to the electro dynamic vibrator 3. This will create an opposite force F_{c} to outbalance the external applied force F, which will render a zero static offset for the spatial position 10 when the correct static force F is applied by the human body 7.

The calibrated DC-current i_{c} may be created within the micro computer system 1, whereafter the signal may be amplified by the amplifier 2 to control the electro dynamic vibrator 3. The system is calibrated by first measuring the spatial position 10 when the system is unloaded, without any applied contact force F. Then a calibration weight is mounted on the probe 8 when the system is still unloaded with no additional force asserted except for the calibration weight. The required DC-current i_{c} is automatically adjusted by the micro computer system 1 so that zero offset is achieved for the spatial position 10, i.e. when the spatial position 10 is the same as when no calibration weight is mounted on the probe 8. At zero offset, the applied DC-current i_{c} is measured and the value is permanently stored in the micro computer system 1. During normal operation, the stored calibrated DC-current i_{c} is added to the electrical current signal to the electro dynamic vibrator 3. The contact force F created by the human body part 7 will cause a static displacement that is measured by the detector 6, which detects the signal emanating from the transmitter 4 passing the optional aperture 5. One or two of these items, the transmitter 4, aperture 5 or the detector 6, may be located directly on the vibrating probe 8, whereas at least one item should be spatially fixed.

The contact force F is equal to the calibration weight when the measured static displacement for the spatial position 10 is zero. A contact force F below, at or above the calibration weight will then be presented on the human feedback device 9 as a “too low value”, “equal to” or a “too high value”, respectively. On the human feedback device 9, the contract force F may be visualized on a bar graph LED array. During normal operation, i.e. when the VPT's are recorded, the spatial position 10 signal is measured from the detector 6. This signal may be processed in any way in the micro computer system 1, i.e. low pass filtered. The filtered signal from the detector 6 can be represented as shown in FIGS. 3 and 4, dependent upon the selected method for monitoring the static skin force F applied by the patient 7. In FIGS. 3 and 4, X₁ corresponds to the spatial position 10 for an unloaded system, and X_{cal} to the spatial position 10 when the calibration weight is mounted on the vibrating probe 8 without added DC-current i_{c}.

The VPT is preferably recorded by reading the real acceleration from the accelerometer sensor mounted directly on the vibrating probe 8. To enhance the accuracy, it is also important to register the current skin temperature since the VPT varies with this parameter. The skin temperature may be measured continuously during the measurement or at least at the beginning just before the start of measurement. The temperature may be measured with a temperature sensor mounted on the vibrating probe 8 or in a separate place elsewhere on the measuring device. Prior to a measurement, the device shall perform a self-calibration to make sure that the required starting conditions prevail. This calibration shall at least include a tare of the spatial position 10, a frequency control to make sure that the used frequencies run within certain limits, and a measurement of background vibration noise. Additionally, the maximum and minimum recordable amplitudes and accelerations may be measured during the self-calibration.

The human feedback device 9 is used to report the measuring status to both the operator and the patient to be tested. Prior to measurement, the device 9 should indicate when it is ready and calibration is finished. During the measurement, the device 9 should show the status for the applied skin contact force F, i.e. if the force is too high, too low or within the required limits. The used “feedback principle” may be a light by using an LED/lamp-array with different colors, a flashing lamp or an LED with different flashing frequencies or some kind of numerical or graphical display to represent the status. An audible feedback signal (speaker or headphones), may be used as a human feedback device 9 where a combination of different frequencies and/or amplitudes are utilized to represent the status.

As an added feature, it is possible to automatically compensate the registered VPT if the applied contact skin force F is outside the required limits, when the force is either too high or too low. For this case, the actual spatial vibration amplitude (mean value) read by the detector 6 is used to compensate for an erroneous contact force F. If the applied contact skin force F is too low in a balanced system, as shown in FIG. 4, the measured mean value X of the spatial position 10 is larger than X₁ The offset X and X₁, can then be converted to a specific acceleration offset value which should be added to the read acceleration in order to get a compensated VPT. The same principle will also work if the applied contact skin force F is too high in a balanced system. In that case, the read offset value X and X₁, will be negative which corresponds to a negative acceleration offset. The read acceleration should then be reduced with the corresponding converted negative acceleration offset in order to get a compensated VPT.

A full test cycle comprises the following steps:
(1) The operator starts a measurement by either pressing a start button or entering a start command to the device.
(2) The device starts with a self-calibration which is displayed on the human feedback device 9. When the self-calibration is finished, the human feedback device 9 reports a ready to measure condition.
(3) The patient to be examined applies the appropriate body part 7, i.e. a finger, on the vibrating probe 8, whereas the human feedback device 9 reports the applied skin force F. At this stage, an integrated temperature sensor on the vibrating probe 8 may measure the skin temperature. Alternatively, the temperature may be measured in a different manner shortly before the body part 7 is placed on the vibrating probe 8.
(4) When the applied skin force F is within the required limits, the probe 8 starts to vibrate in a predetermined ascending sequence.
(5) When the patient feels a vibration, he or she presses an external button which will switch the vibration to a descending sequence. During the descending sequence, the patient continues to press the external button until he or she does not feel vibrations anymore.
(6) When the patient releases the external button, when no further vibration is felt, the device will switch back to an ascending sequence and the procedure jumps back to point 5 above, and so on, until a full test sequence is completed. A completed test sequence includes changes in the vibration frequencies according to a well-defined scheme.
(7) The vibration excitation stops when the VPT's have been registered for all required frequencies. The recorded VPT's may then be compared with normative data from a healthy person. The result may be reported to the operator as an SI value which is an absolute figure telling if the patient is healthy or not, in terms of neuropathy.

During the test cycle, according to points 5 and 6 above, the applied skin contact force F is monitored continuously by reading the spatial position 10. The read spatial position 10 is converted to a contact force F which is continuously displayed on the human feedback device 9. The patient reads the output and adjusts the contact force F accordingly. The device 9 may calculate an internal compensation to adjust the recorded VPT if the patient does not make any adjustments or if adjustments are insufficient. The VPT's are recorded as the mean value of the read max and min acceleration (rms values) during the ascending and descending cycle.

In an unbalanced system, when the correct contact force F is applied by the patient 7, the offset for the DC-component in the spatial position 10 signal shall be equal to X₁ and X_{cal}. If the measured offset is higher, then the patient must decrease the applied skin-force F and vice versa, i.e. increase the applied skin-force F if the offset is too low. With this method, no added DC-current component i_{c} is necessary in the electrical signal which drives the electro dynamic vibrator 3.

In a balanced system, a DC-current i_{c} is added to the electrical signal which drives the electro dynamic vibrator 3. When this current is added and when the correct contact force F is applied by the patient 7, the DC-component in the spatial position 10 signal shall be equal to X₁, which corresponds to a zero static offset. If the measured spatial position 10 is less than X₁, then the patient 7 must decrease the applied skin-force F and vice versa, i.e. increase the applied skin-force F if the spatial position 10 is larger than X₁

For spatial detection, the spatial position 10 can be measured in many ways, but the basic principle is that the vibrating probe 8 is moved when the human body part 7 applies a force F on the probe 8. The spatial position 10 and the subsequent movement will alter the signal from the transmitter 4, and the detector 6 measures this spatial alteration of the transmitted signal. In this respect, the transmitter 4 can be mounted directly on the vibrating probe 8, while the detector 6 is fixed in space. Alternatively, the detector 6 may be mounted directly on the vibrating probe 8, while the transmitter 4 is fixed in space. As a second alternative, both the transmitter 4 and the detector 6 are fixed in space, whereas, the aperture 5 is mounted directly on the vibrating probe 8. The combination of the transmitter 4 and the detector 6 makes a matched pair that can use different techniques. The following are some examples of the different techniques used.

| Transmitter | Detector |
|---|---|
| Light Emitting Diode, LED | Position Sensitive Detector |
| | (PSD) |
| LASER Diode | Position Sensitive Detector |
| | (PSD) |
| Light Emitting Diode, LED | Photo Detector |
| LASER Diode | Photo Detector |
| Permanent Magnet | Magnetic Field Sensor |
| Electro Magnet | Magnetic Field Sensor |

Having thus described the present invention, what is desired to be obtained for Letters Patent is:

1-6. (canceled)

7. In a method for testing or screening of peripheral neuropathies at a skin site of a subject, the method employing an apparatus having a support device for supporting a body part containing the skin site of a subject to be tested, a vibration generating device having a contact element for positioning in the skin site, a frequency generating device connected to the vibration generating device, a control circuit, a switch, and a human feedback device, the improvement in the method comprising:
controlling contact between the vibration generating device and the skin site;
vibrating the vibration generating device at specific predetermined frequencies;
supplying a known discrete frequency signal between the frequency generating device and vibration generating device;
modifying amplitude of the frequency signal controlling the circuit in an ascending and descending mode by a subject actuating the switch providing response signals to the control circuit; and
obtaining a threshold signal value from the response signals keeping continuous contact force between the skin site and contact within a predefined range.

8. The method according to claim 7, further comprising:
providing a transmitter transmitting the frequency
signal;
providing a receiver measuring a spatial position alteration of a transmitted signal; and
providing a micro computer system automatically adjusting a DC-current achieving zero offset for the spatial position.

9. The method according to claim 8, wherein the transmitter is transmitting light emission.

10. The method according to claim 8, wherein the transmitter is transmitting an electromagnetic field.

11. The method according to claim 8, wherein the receiver operates by photo electric detection.

12. The method according to claim 8, wherein the receiver operates by magnetic field sensing.

13. The method according to claim 7, wherein continuous contact force between the skin site and vibrating device is automated by providing a feedback force compensation unit.

14. The method according to claim 7, wherein a vibrotactile perception threshold is recorded by reading an acceleration from an accelerometer.

15. The method according to claim 7, wherein the human feedback device uses a feedback principle based on LED or lamp arrays to provide a graphical or numerical display.

16. The method according to claim 7, wherein a temperature is measured at the skin site prior to and during the entire testing.
